(19) United States
(12) Patent Application Publication
(10) Pub. No.: US 2024/0138673 A1
(43) Pub. Date: May 2, 2024

(54) Switched Light Source Microlens Array (SLSMA) for Retina Projection

(71) Applicant: GlaiveRF, Inc., Carlisle, MA (US)

(72) Inventors: Wayne H. Woods, JR., Carlisle, MA (US); Christopher Shelby, Shreveport, LA (US)

(21) Appl. No.: 18/384,585

(22) Filed: Oct. 27, 2023

Related U.S. Application Data
(60) Provisional application No. 63/420,145, filed on Oct. 28, 2022.

(51) Int. Cl. A61B 3/14; A61F 2/16

(52) U.S. Cl. CPC A61B 3/14; A61F 2/1613

(57) ABSTRACT

A surgically implanted ocular optical array that can be used in both therapeutic and diagnostic applications is described. A device configured to be implanted in an eye includes: an imaging system that receives visible light incoming to the eye; and a light generation panel and a microlens array that are configured to generate and project an image onto a retina of the eye in which the device is implanted, the image being based on the light received by the imaging system

### RELATED APPLICATIONS

This application claims priority to U.S. provisional application No. 63/420,145 filed Oct. 28, 2022, the content of which is incorporated by reference herein in its entirety.

### BACKGROUND

The present invention relates generally to ocular implants and, more particularly, to surgically implanted ocular optical array that can be used in both therapeutic and diagnostic applications.

Being able to target/stimulate specific areas of the retina surface is desirable and difficult to achieve. Approaches to doing this have included chips that directly interface with the neurons in the retina surface. In this disclosure, devices and methods are described that are much less surgically invasive compared to such alternatives.

### SUMMARY

In an aspect of the invention, there is a device configured to be implanted in an eye, the device comprising: an imaging system that receives visible light incoming to the eye; and a light generation panel and a microlens array that are configured to generate and project an image onto a retina of the eye in which the device is implanted, the image being based on the light received by the imaging system.

In an embodiment, the device further comprises control circuitry that causes the light generation panel and the microlens array to project the image onto a determined area of the retina.

In an embodiment, the microlens array comprises an array of optical lenses and the light generation panel comprises a plurality of individually controllable light emitting elements.

In an embodiment, the determined area of the retina is a healthy area of the retina.

In an embodiment, the control circuitry determines the determined area of the retina using a stored mapping.

In an embodiment, the imaging system, the control circuitry, the light generation panel, and the microlens array are arranged in a chip stack.

In an embodiment, the imaging system is at a first side of the chip stack, the microlens array is at a second side of the chip stack opposite the first side of the chip stack.

In an embodiment, the device comprises a body comprising a central portion and tabs extending outward from the central portion, and the chip stack is in the central portion.

In an embodiment, the device further comprises a wireless communication antenna that is configured to receive wireless communication signals from outside the device.

In an embodiment, the control circuitry is configured to program the mapping based on the wireless communication signals.

In an embodiment, the device further comprises a rechargeable battery that is configured to power the imaging system, the control circuitry, and the light generation panel.

In an embodiment, the rechargeable battery is configured to be recharged wirelessly from a charging system located outside the eye.

In an embodiment, the device is configured to be implanted in a capsular bag of the eye.

In an embodiment, the device is configured to be implanted in a ciliary sulcus of the eye.

In an embodiment, the device is configured to be implanted in a chamber of the eye anterior to the iris.

In an embodiment, a method comprises implanting the device into the eye.

In an embodiment, a method of using the device comprises: causing the device to project a diagnostic image on different locations of the retina of the eye; receiving patient feedback for each of the different locations; creating a mapping of the retina of the eye based on the feedback; and programming the mapping into the device.

In an embodiment, the method of using the device comprises optimizing the mapping using artificial intelligence.

In an embodiment of the method of using the device, the mapping maps the retina into functional areas and non-functional areas.

In an embodiment of the method of using the device, the device is configured to control one or more elements of the light generation panel based on the mapping to project an image onto a functional area of the retina to reduce or eliminate a scotoma caused by a non-functional area of the retina.

In an embodiment, a device according to any of the aspects above comprises a body made of acrylic and/or silicone lens material.

In an embodiment, a device according to any of the aspects above comprises a single piece lens.

In an embodiment, a device according to any of the aspects above comprises a body having dimensions of 1 mm<=TH<=3 mm and 1 mm<=W<=10 mm.

In an embodiment, a device according to any of the aspects above comprises an imaging chip comprising the imaging system, a control chip comprising the control circuitry, a chip comprising the light generation panel, and a microlens chip comprising the microlens array, wherein the chips are arranged in a chip stack. The chips may be made using semiconductor fabrication materials and techniques, including but not limited to Si, InP, GaAs, Liquid Crystal materials, and BGA/C4/micro-BGA, through substrate (or silicon) vias (TSVs), micro-TSVs, and solder or oxide bonding techniques.

In an embodiment, a device according to any of the aspects above comprises a wireless communication antenna (e.g., for receiving programming signals) and/or an inductive coupling coil (e.g., for wireless charging) embedded in the material of the body.

### BRIEF DESCRIPTION OF THE DRAWINGS

The present invention is described in the detailed description which follows, in reference to the noted plurality of drawings by way of non-limiting examples of exemplary embodiments of the present invention.

FIG. 1 shows a diagram of a healthy eye.

FIG. 2 shows a diagram of a damaged eye.

FIG. 3A shows a diagram of a healthy retina corresponding to the eye of FIG. 1.

FIG. 3B shows a diagram of a damaged retina corresponding to the eye of FIG. 2.

FIG. 4A shows a diagram of an image that is incident on the healthy retina of FIG. 3A and the resulting view to the person.

FIG. 4B shows a diagram of an image that is incident on the damaged retina of FIG. 3B and the resulting view to the person.

FIG. 5 shows an embodiment of a microlens device implanted in a capsular bag of an eye in accordance with aspects of the invention.

FIG. 6 shows an embodiment of a microlens device implanted in a ciliary sulcus of an eye in accordance with aspects of the invention.

FIG. 7 shows an embodiment of a microlens device implanted in an anterior chamber of an eye in accordance with aspects of the invention.

FIG. 8 shows a diagram of an exemplary projection of an image on a retina by a microlens device in accordance with aspects of the invention.

FIG. 9 shows a diagram of an exemplary projection of an image on a retina by a microlens device in accordance with aspects of the invention.

FIG. 10 shows a diagram of an image projected on the healthy area of the retina by a microlens device in accordance with aspects of the invention, and a view of what the person sees based on the image being projected in the manner shown.

FIG. 11 shows an exemplary point-to-point pixelated mapping from a 2D directionally programmable optical array to a retina optical nerve surface in accordance with aspects of the invention.

FIG. 12 shows an exemplary point-to-point pixelated mapping from a 2D directionally programmable optical array to a retina optical nerve surface in accordance with aspects of the invention.

FIG. 13 shows a flowchart of an exemplary method in accordance with aspects of the invention.

FIG. 14A shows exemplary locations on eyeglasses for coils that may be used to wirelessly charge the microlens device in accordance with aspects of the invention.

FIG. 14B shows an exemplary location on an eyepatch for coils that may be used to wirelessly charge the microlens device in accordance with aspects of the invention.

FIGS. 15A and 15B shows an example of a microlens array and light projection panel in accordance with aspects of the invention.

FIG. 16 shows an embodiment of a microlens device in accordance with aspects of the invention.

FIG. 17A shows a flowchart of an exemplary method in accordance with aspects of the invention.

FIG. 17B shows a coarse grid used in the method of FIG. 17A.

FIG. 17C shows a fine grid used in the method of FIG. 17A.

FIGS. 18A and 18B show a side view and a top view, respectively, of a microlens device in accordance with aspects of the invention.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

The present invention relates generally to ocular implants and, more particularly, to surgically implanted ocular optical array that can be used in both therapeutic and diagnostic applications. In embodiments, a device comprises an optical array, preferably microlens array, integrated to control electronics and charged-coupled device (CCD)/electronic cameras. In embodiments, a camera is integrated in a single assembly with the implanted microlens array. In this way, when the device is implanted in an eye of a patient, the patient has vision which tracks with eyeball direction as opposed to, for example, a camera system mounted on a pair of glasses and communicated to the microlens array from a wired/tethered or wireless network bridge.

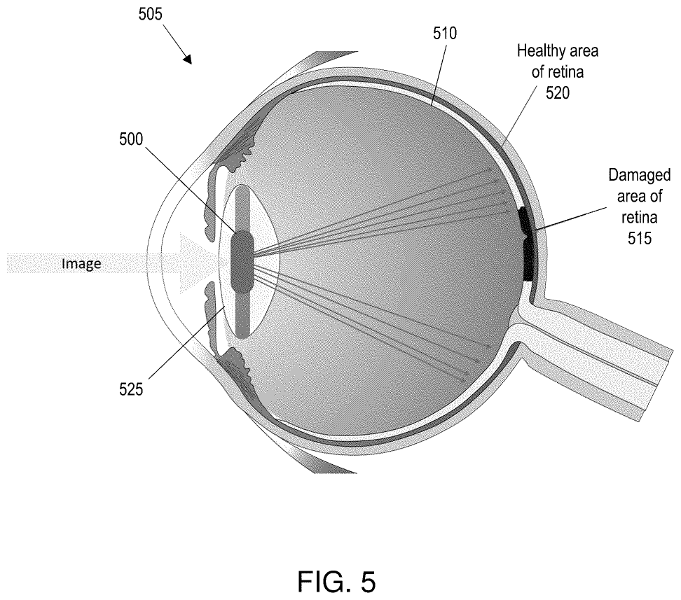

In an embodiment, the camera, optical signal sources, control electronics, programmable optical array, and power source (e.g., batteries) are all integrated in one device which is surgically implanted in the eye as shown. Exemplary embodiments of implants are shown in FIGS. 5-7.

In embodiments, the surgically implanted chip is wirelessly powered via an inductively coupled primary coil that can be positioned at various locations near the implanted chip, such as for example, on a pair of glasses or on a monocle-style mounting.

Devices according to aspects of the invention allow visible light probing of the retina, including the extreme periphery of functioning retinal tissue. A microlens array implementation of the optical array is well-suited for this application because it has no moving parts.

In embodiments, very detailed (e.g., micron-scaled) maps of functional and non-functional areas of the retina are made by probing/testing precise areas of the retina using an implant in accordance with aspects of the invention.

By being able to probe/test precise areas of the retina, detailed, maps of the functional retina tissue can be created. This mapping provides an advantage over devices that do not utilize mapping, since the mapping permits the inventive devices to precisely target light onto functional areas of the retina. In embodiments, a device is implanted near the front of the eye. This type of surgery is much less invasive and problematic than trying to implant a chip with an array of electrical needle probes or chemical injection ports directly onto the retina surface. Embodiments thus provide a much more practical approach and will allow many more doctors to be able to be trained for the procedure which would be similar to other common surgical eye procedures/implants.

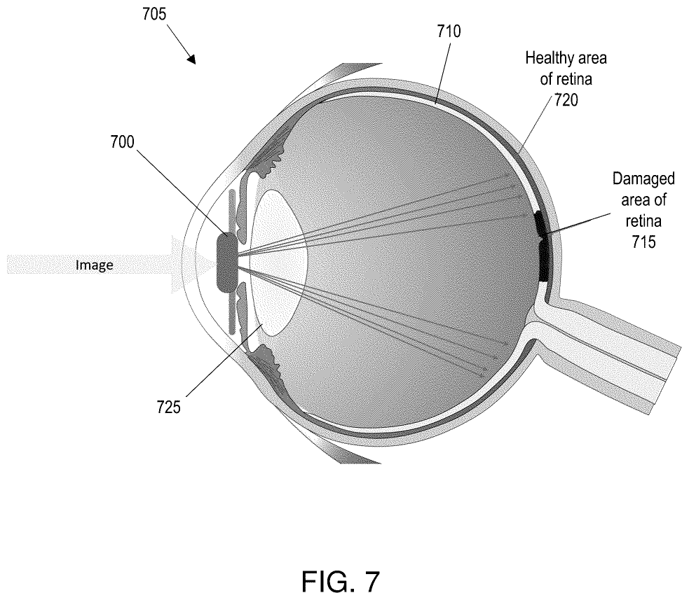

In one embodiment, a wirelessly powered and programmable device including an integrated CCD, control electronics, and microlens is surgically implanted in the eyeball as shown, for example, in FIG. 7. In one example, the device is hermetically sealed and completely self-contained.

Devices according to aspects of the invention may be used diagnostically, e.g., for creating detailed functional retinal tissue maps. Devices according to aspects of the invention may be used therapeutically, e.g., for image construction and projection onto functional retinal tissue in real time.

In embodiments, there is a surgically implanted integrated device that includes a camera, control circuitry, a light generation panel, and a microlens array for retinal image generation. In embodiments, the device is used for mapping healthy (also called functional) retina tissue and unhealthy (also called damaged or non-functional) retina tissue. In embodiments, the device is used for image projection onto healthy retina tissue. In embodiments, the device is used to project eyeball-motion directed images selectively onto the healthy portions of retina tissue according to a map. The device may have wirelessly powered variants. The device may be used to perform a method of mapping healthy and unhealthy areas of the retina.

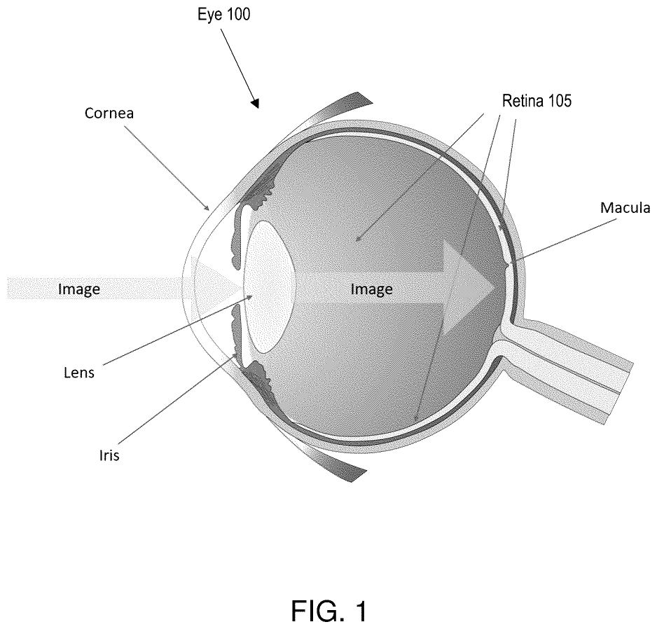

FIG. 1 shows a diagram of a healthy eye 100. As shown in FIG. 1, an image in the form of visible light enters the cornea and is focused onto the lens and then finally onto the macula e.g., (central portion of the retina 105) which allows for clear vision.

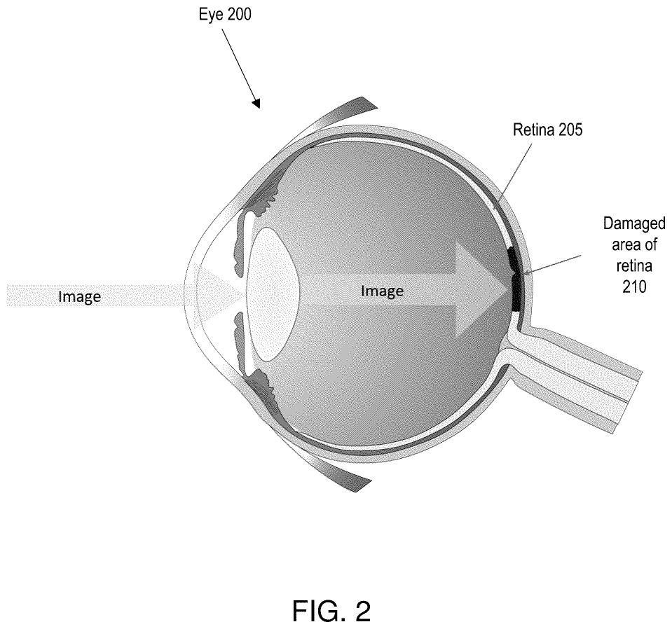

FIG. 2 shows a diagram of a damaged eye 200. As shown in FIG. 2, retinal scarring in the macula (e.g., macular degeneration) results in a damaged area 210 of the retina 205 that causes loss of central vision (e.g., scotoma). Implementations of the invention seek to take advantage of the still healthy areas of the retina, e.g., not including the damaged area, to help patients regain significant visual function.

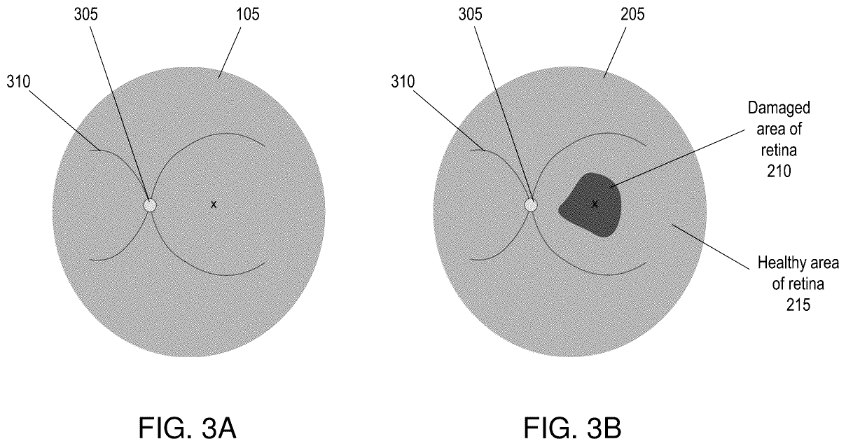

FIG. 3A shows a diagram of a healthy retina 105 corresponding to the eye 100 of FIG. 1. Also shown are the optic nerve/disc 305 and retinal veins/arteries 310. As shown in FIG. 3A, the retina 105 does not have a damaged portion and, thus, provides normal central vision for the person.

FIG. 3B shows a diagram of a damaged retina 205 corresponding to the eye 200 of FIG. 2. Also shown are the optic nerve/disc 305 and retinal veins/arteries 310. As shown in FIG. 3B, the retina 205 includes a damaged area 210 that produces a large central scotoma in the person's vision. As shown in FIG. 3B, the retina 205 includes undamaged area 215 around the damaged area 205.

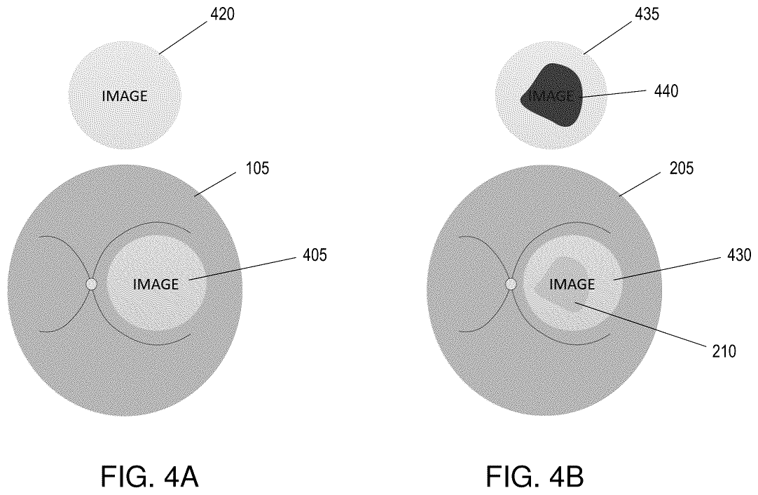

FIG. 4A shows a diagram of an image 405 (e.g., visible light) that is incident on the healthy retina 105 of FIG. 3A. FIG. 4A illustrates a view 420 of what this person sees based on the image 405 being incident on the retina 105. As shown in FIG. 4A, the view 420 of what this person sees is a normal view without any scotoma.

FIG. 4B shows a diagram of an image 430 (e.g., visible light) that is incident on the damaged retina 205 of FIG. 3B. FIG. 4B illustrates a view 435 of what this person sees based on the image 430 being incident on the retina 205 having the damaged area 210. As shown in FIG. 4B, the view 435 of what this person sees has a large central scotoma 440, represented in this case by a dark or fuzzy spot in an otherwise normal view.

FIG. 5 shows an embodiment of a microlens device 500 implanted in a capsular bag of an eye in accordance with aspects of the invention. As shown in FIG. 5, the eye 505 includes a retina 510 that has a damaged area 515 and a healthy area 520, e.g., similar to the retina 205 shown in FIGS. 3B and 4B. The eye 505 also includes a capsular bag 525 which normally contains the lens, e.g., the human crystalline lens. In accordance with aspects of the invention, the human lens is removed and replaced with the microlens device 500 that is configured to receive an image (e.g., visible light) from outside the eye and project the image onto the healthy area 520 of the retina 510 using directional projection by a microlens array in the microlens device 500. By projecting the image onto the healthy area 520 of the retina 510 and avoiding projecting the image onto the damaged area 515, the implanted microlens device 500 provides this person with a view that eliminates or greatly reduces the scotoma that this person would otherwise have if the microlens device 500 were not present.

The microlens device 500 may be implanted in the capsular bag 525 after primary cataract surgery or as an intraocular lens exchange with intact posterior capsule. An exemplary method for implanting the microlens device 500 in the capsular bag 525 includes: making a 6-8 mm incision at the limbus or slightly posterior (1-2 mm) posterior to the limbus; through a pharmacologically dilated pupil, making a 6-8 mm diameter opening in the anterior capsular bag; and removing the human crystalline lens entirely in an extra capsular fashion such as phacoemulsification. If the eye is pseudophakic with an intact posterior capsule, then intraocular lens is dissected free of its capsular attachment and removed from the eye. The capsular opening is then widened if necessary. The microlens device 500 is then placed through the primary incision and into the capsular bag. The haptics of the microlens device 500 keep the implant centered in the capsular bag as it heals and creates a fibrotic membrane to stabilize the implant and place the microlens device 500 directly in the visual axis for the purpose of projecting the central image onto the healthiest part of the retina as close to the damaged area 515 as possible. In embodiments where the microlens device 500 has external wiring, the wires coming from the microlens device 500 may be placed anterior to the anterior capsule and posterior to the iris and routed to the limbus, for example, through a 30 or 27 gauge temporal sclerotomy 2-3 mm posterior to the limbus. The wires may be left subconjunctival to prevent foreign body sensation. All support material may be removed, and the primary wound may be closed with sutures if needed. The microlens device 500 is thus held inside the capsular bag 525. Over time, the bag fibrosis around the haptics of the implant is stable in place.

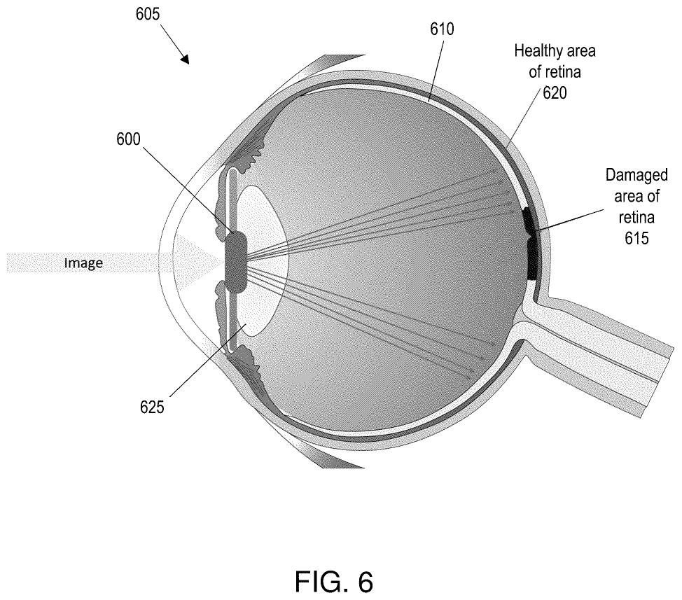

FIG. 6 shows an embodiment of a microlens device 600 implanted in a ciliary sulcus of an eye in accordance with aspects of the invention. The ciliary sulcus is a small space between the posterior surface of the iris base and the anterior surface of the ciliary body. As shown in FIG. 6, the eye 605 includes a retina 610 that has a damaged area 615 and a healthy area 620, e.g., similar to the retina 205 shown in FIGS. 3B and 4B. The eye 605 also includes a capsular bag 625 which normally contains the lens, e.g., the human crystalline lens. In accordance with aspects of the invention, the human lens is removed and the microlens device 600 is inserted into the ciliary sulcus. The microlens device 600 is configured to receive an image (e.g., visible light) from outside the eye and project the image onto the healthy area 620 of the retina 610 using directional projection provided by a microlens array in the microlens device 600. By projecting the image onto the healthy area 620 of the retina 610 and avoiding projecting the image onto the damaged area 615, the implanted microlens device 600 provides this person with a view that eliminates or greatly reduces the scotoma that this person would otherwise have if the microlens device 600 were not present.

The microlens device 600 may be implanted in the ciliary sulcus after primary cataract surgery with compromised posterior capsule or as an intraocular lens exchange with open posterior capsule. An exemplary method for implanting the microlens device 600 in the ciliary sulcus includes: making a 6-8 mm incision at the limbus or slightly posterior (1-2 mm) posterior to the limbus; through a pharmacologically dilated pupil, making a 6-8 mm diameter opening in the anterior capsular bag; and removing the human crystalline lens entirely in an extra capsular fashion such as phacoemulsification. A thorough anterior vitrectomy is performed in the presence of a posterior capsule defect. If the eye is pseudophakic with an open posterior capsule, the intraocular lens is dissected free of its capsular attachment and removed from the eye. The capsular opening is then widened if necessary and a thorough anterior vitrectomy is performed. The microlens device 600 is placed through the primary incision and into the ciliary sulcus on the anterior aspect of the capsular bag, directly posterior to the iris. The haptics of the microlens device 600 will keep the implant centered in the ciliary sulcus to stabilize the implant and place the microlens device 600 directly in the visual axis for the purpose of projecting the central image onto the healthiest part of the retina as close to the damaged area 615 as possible. In embodiments where the microlens device 600 has external wiring, the wires coming from the microlens device 600 may be placed anterior to the anterior capsule and posterior to the iris and routed to the limbus, for example, through a 30 or 27 gauge temporal sclerotomy 2-3 mm posterior to the limbus. The wires may be left subconjunctival to prevent foreign body sensation. All support material may be removed, and the primary wound may be closed with sutures if needed. The microlens device 600 haptics rest in the ciliary sulcus posterior to the iris and directly anterior to the capsular bag, which stabilizes the lens.

FIG. 7 shows an embodiment of a microlens device 700 implanted in an anterior chamber of an eye in accordance with aspects of the invention. As shown in FIG. 7, the eye 705 includes a retina 710 that has a damaged area 715 and a healthy area 720, e.g., similar to the retina 205 shown in FIGS. 3B and 4B. The eye 705 also includes a capsular bag 725 which normally contains the lens, e.g., the human crystalline lens. In accordance with aspects of the invention, the human lens is removed and the microlens device 700 is inserted into the anterior chamber of an eye, e.g., anterior to the iris. The microlens device 700 is configured to receive an image (e.g., visible light) from outside the eye and project the image onto the healthy area 720 of the retina 710 using image projection provided by a microlens array in the microlens device 700. By projecting the image onto the healthy area 720 of the retina 710 and avoiding projecting the image onto the damaged area 715, the implanted microlens device 700 provides this person with a view that eliminates or greatly reduces the scotoma that this person would otherwise have if the microlens device 700 were not present.

The microlens device 700 may be implanted in the anterior chamber after primary cataract surgery with no capsular support or as an intraocular lens exchange with no capsular support. An exemplary method for implanting the microlens device 700 in the anterior chamber includes: making a 6-8 mm incision at the limbus or slightly posterior (1-2 mm) posterior to the limbus; through a pharmacologically dilated pupil, making a 6-8 mm diameter opening in the anterior capsular bag; and removing the human crystalline lens entirely in an extra capsular fashion such as phacoemulsification. A thorough anterior vitrectomy is performed in the absence of sufficient capsular support. If the eye is pseudophakic with an open posterior capsule, the intraocular lens is dissected free of its capsular attachment and removed from the eye, and a thorough anterior vitrectomy is performed in the absence of sufficient capsular support. Miosis of the pupil may be performed to provide support for the microlens device 700. The microlens device 700 is then placed through the primary incision and into the anterior chamber directly anterior to the iris. The haptics of the microlens device 700 are seated into the anterior chamber angle to stabilize the implant and place the microlens device 700 directly in the visual axis for the purpose of projecting the central image onto the healthiest part of the retina as close to the damaged area 715 as possible. A small peripheral iridotomy may be performed to prevent pupillary block. In embodiments where the microlens device 700 has external wiring, the wires coming from the microlens device 700 may be placed anterior to the anterior capsule and posterior to the iris and routed to the limbus, for example, through a 30 or 27 gauge temporal sclerotomy 2-3 mm posterior to the limbus. The wires may be left subconjunctival to prevent foreign body sensation. All support material may be removed, and the primary wound may be closed with sutures if needed.

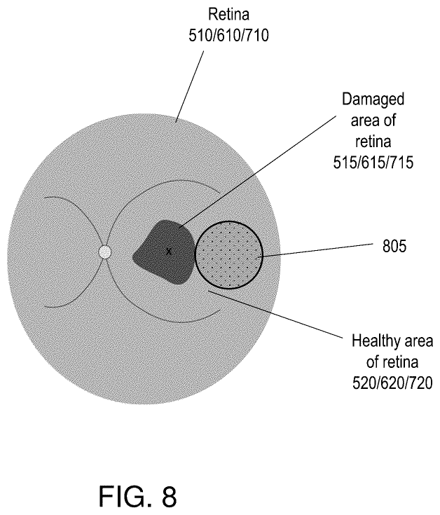

FIG. 8 shows a diagram of an exemplary projection of an image on a retina 510/610/710 by a microlens device 500/600/700 in accordance with aspects of the invention. As described with respect to FIGS. 5-7, the microlens device 500/600/700 receives an incoming image, in the form of visible light from outside the eye, and projects the image onto a healthy area 520/620/720 of the retina while avoiding projecting the image onto the damaged area 515/615/715. FIG. 8 shows the projection area 805 relative to the damaged area 515/615/715. The shape of the projection area 805 in FIG. 8 is illustrative, and the projection area 805 may have other shapes different than what is shown in FIG. 8.

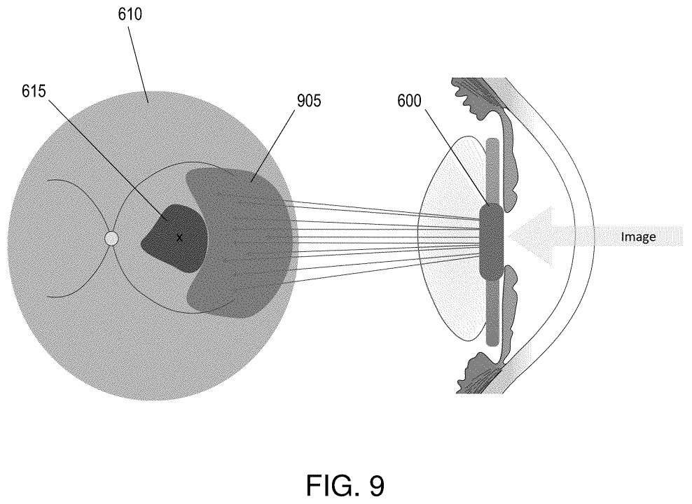

FIG. 9 shows a diagram of an exemplary projection of an image on a retina by a microlens device 600 in accordance with aspects of the invention. As described with respect to FIG. 6, the microlens device 600 receives an incoming image, in the form of visible light from outside the eye, and projects the image onto a healthy area of the retina while avoiding projecting the image onto the damaged area 615. FIG. 9 shows the projection area 905 relative to the damaged area 615. Specifically, the microlens device 600 takes the central image and shifts the projection onto the adjacent healthy area of the retina. In this manner, the healthy area of the retina adjacent to the damaged area of the retina can be used for central vision. The shape of the projection area 905 in FIG. 9 is illustrative, and the projection area 905 may have other shapes different than what is shown in FIG. 9. Although FIG. 9 only shows the microlens device 600, it should be understood that the microlens device 500 and the microlens device 700 may function in a similar manner, with a difference being where the different devices 500, 600 700 are implanted in the eye.

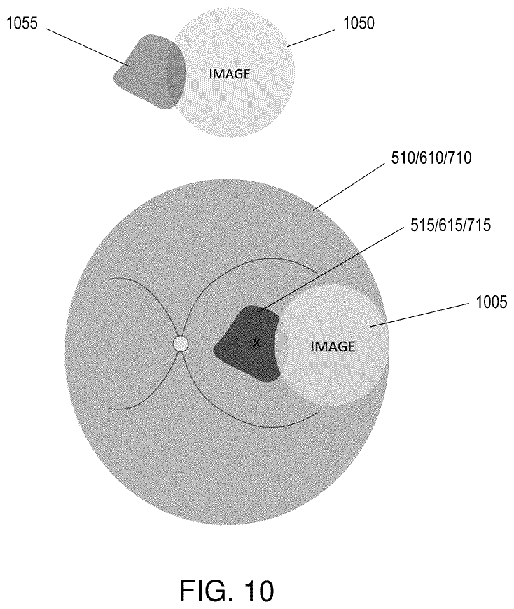

FIG. 10 shows a diagram of an image 1005 projected on the healthy area of the retina 510/610/710 by a microlens device 500/600/700 in accordance with aspects of the invention, and a view 1050 of what the person sees based on the image being projected in the manner shown. As described herein, the microlens device 500/600/700 projects the image onto the healthy area of the retina adjacent to the damaged area 515/615/715 of the retina. In this manner, the view 1050 of what the person sees has the scotoma 1055 shifted away from the center, such that the person can now see central visual details unimpeded by the scotoma. Comparing the view 1050 of FIG. 10 to the view 435 of FIG. 4B, it is evident that the microlens device 500/600/700 provides a vast improvement in central vision for the person.

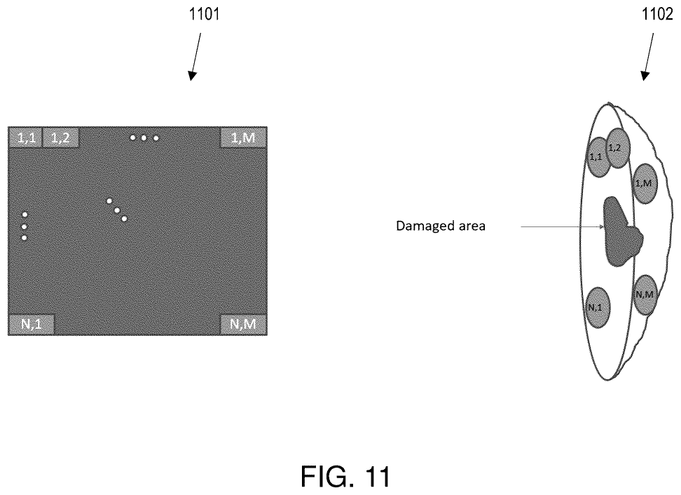

FIG. 11 shows an exemplary point-to-point pixelated mapping from a 2D directionally programmable optical array 1101 to a retina 1102 optical nerve surface in accordance with aspects of the invention. In embodiments, individual elements of the array 1101 are mapped to locations on the retina 1102. The microlens device 500/600/700 may use the mapping defined in the array to control the projection of the image onto healthy areas of the retina and avoid projecting onto the damaged areas of the retina.

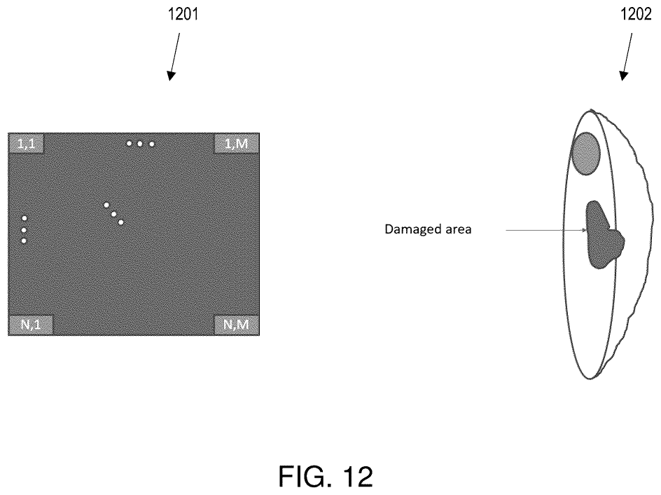

FIG. 12 shows an exemplary point-to-point pixelated mapping from a 2D directionally programmable optical array 1201 to a retina 1202 optical nerve surface in accordance with aspects of the invention. In embodiments, the microlens device 500/600/700 produces a moving spot that is dynamically swept across retina. The microlens device may project a spot size that can be used to create high-definition quality pixel sizes on the retina. The visible wavelength is between about 380 nm and 750 nm and the eyeball is about 1 to 2 inches long, which is about 25 mm to 50 mm, which is about 30 to 130 wavelengths long, which means that the image projected by the microlens device 500/600/700 can be close to the near field.

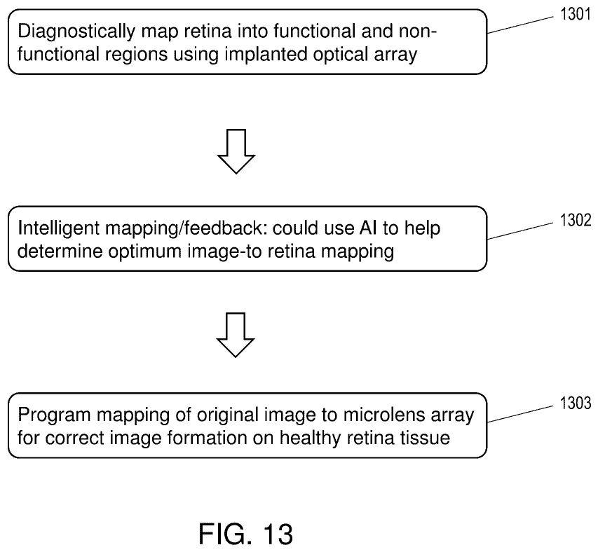

FIG. 13 shows a flowchart of an exemplary method in accordance with aspects of the invention. At step 1301, the implanted microlens device 500/600/700 is used to diagnostically map a retina (of the eye in which the device is implanted) into functional and non-functional regions, e.g., healthy areas and damaged areas. This may include projecting an image onto a mapped location on the retina and receiving feedback from the person as to whether they can or cannot see the image clearly. This is repeated for all locations on the 2D array that are mapped to locations on the retina. In this manner, the implanted microlens device 500/600/700 can be used to map the areas of the retina.

Step 1302 comprises using artificial intelligence to optimize the mapping that was determined at step 1301. The shape of the damaged areas and healthy areas of each person's retina will be unique and irregular. In embodiments, an optimum mapping of a regular 2D grid array of input pixels to the irregular healthy regions is determined using artificial intelligence. For example, an artificial neural network may be used to optimize a map of the regular input pixel grid to the irregular healthy retinal tissue, while minimizing the radius from the center of the retina, and while also seeking to maximize the symmetry of the pixel projection around the center. These sorts of constrained mapping tasks are well suited for AI in general and artificial neural networks specifically. The mapping here may take into account complex procedures using artificial neural networks that not only map to healthy retina tissue, but also take into account brain plasticity for image reconstruction.

Step 1303 involves program mapping of an original image to the optical array for correct image formation on the healthy area of the retina. In embodiments, the array that defines the mapping is stored in a programmable circuit of the microlens device 500/600/700. In embodiments, when in use, the microlens device 500/600/700 uses the mapping defined in the array to selectively control an array of light emitters in a light generation panel to project the image onto the healthy areas of the retina as defined in the mapping.

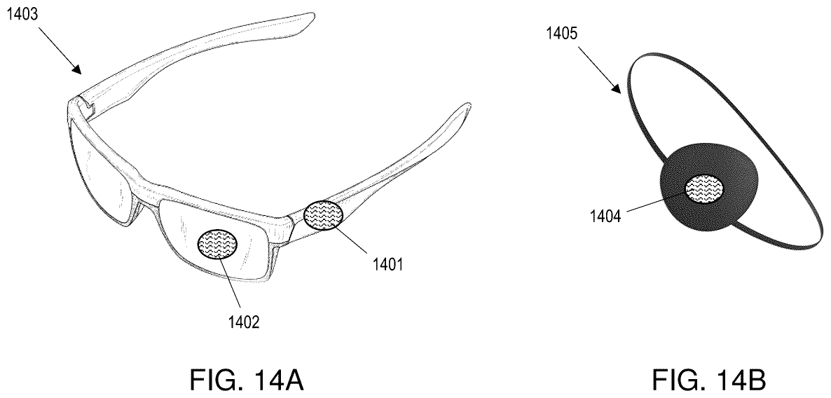

FIG. 14A shows exemplary locations 1401, 1402 on eyeglasses 1403 for coils that may be used to wirelessly charge the microlens device 500/600/700 that is implanted in a person's eye. FIG. 14B shows an example of a location 1404 on an eyepatch 1405 for coils that may be used to wirelessly charge the microlens device 500/600/700 that is implanted in a person's eye. The external charging system is not limited to eyeglasses or an eye patch, and can be on other devices, such as a contact lens. The external charging system itself can be rechargeable. For example, a contact lens may comprise a battery that is wirelessly rechargeable, e.g., from a docking station, and that same contact lens can include control circuitry and charging coils that utilize power from the battery in the contact lens to inductively charge the microlens device 500/600/700 when the contact lens is within range of the microlens device.

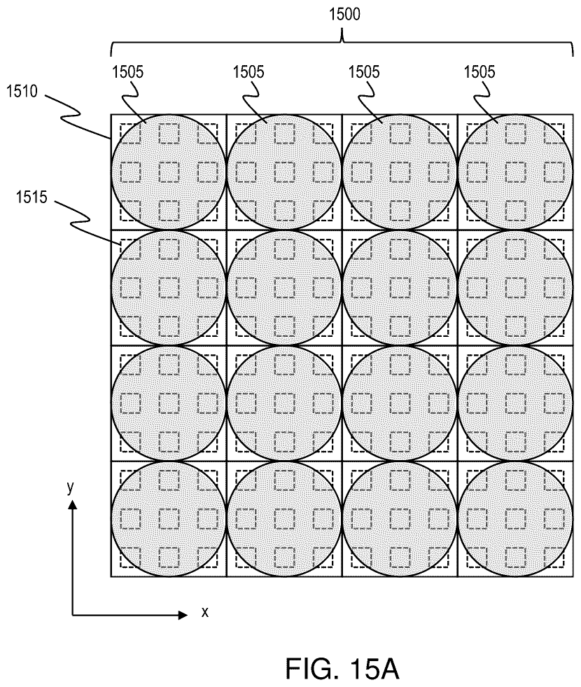

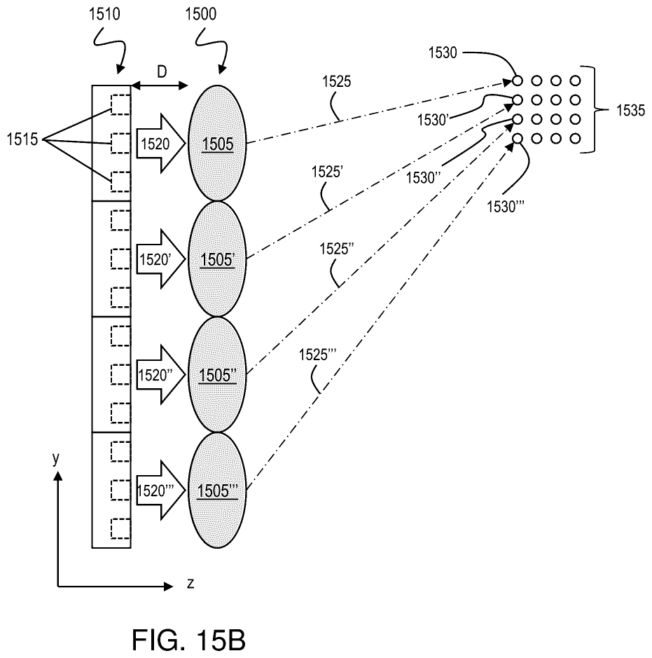

FIG. 15A shows an exemplary microlens array 1500 in accordance with aspects of the invention. In embodiments, the microlens array 1500 comprises an array of plural lenses 1505. The example shown in FIG. 15A includes sixteen lenses 1505 (only four of which are numbered for brevity) arranged in a square-shaped array having four rows and four columns. Each lens 1505 comprises a micron-scale optical lens. In one example, the diameter of each lens 1505 (i.e., in the x direction shown in FIG. 15A) is 100 μm (micrometers), although other lenses 1505 having other dimensions may be used. The shape of each lens 1505 may be circular, square, rectangular, hexagonal, or other shapes. The array of lenses 1505 (i.e., the microlens array) can be arranged in a square, hexagon, or other shape. The array of lenses 1505 (i.e., the microlens array) can have any number of lenses 1505 arranged in any number of rows and columns. The number of rows need not equal the number of columns. A microlens array comprising a plurality of lenses 1505 may be formed using conventional fabrication techniques such as, for example, patterning the array from a substrate of optical material such as glass, quartz, tantalum, zinc selenide, silicon, calcium fluoride, or polymethyl methacrylate.

FIG. 15A also an exemplary light generation panel 1510 arranged behind the microlens array 1500. In embodiments, the light generation panel 1510 includes light emitting elements 1515, each of which may comprise an individually controlled light emitting diode, for example. In embodiments, the light generation panel 1510 includes a number of sections equal to the number of lenses 1505 in the microlens array 1500, with respective ones of the sections aligned with respective ones of the lenses 1505. In embodiments, each of the sections includes a plurality of the light emitting elements 1515, such that a respective plurality of light emitting elements 1515 is associated with each respective one of the lenses 1505. The example shown in FIG. 15A includes nine light emitting elements 1515 per section of the light generation panel 1510 (and thus nine light emitting elements 1515 per lens 1505). However, other numbers of light emitting elements 1515 may be included in each of the sections of the light generation panel 1510, with larger numbers of light emitting elements 1515 providing greater resolution.

Still referring to FIG. 15A, the light generation panel 1510 may be fabricated using semiconductor materials and semiconductor fabrication techniques. In embodiments, the light generation panel 1510 includes a substrate that supports or contains the light emitting elements 1515 and circuitry connected to the light emitting elements 1515. The circuitry may be used for selectively providing electrical power to individual ones of the light emitting elements 1515, such that the light emitting elements 1515 can be individually energized and de-energized using the circuitry. In this manner, the light emitting elements 1515 can be individually turned on (i.e., emitting light) and off (i.e., not emitting light).

FIG. 15A shows the microlens array 1500 and light generation panel 1510 looking along a z axis that is orthogonal to a y axis and an x axis as shown in FIG. 15A. FIG. 15B shows the microlens array 1500 and light generation panel 1510 looking along the x axis that is orthogonal to the y axis and the z axis as shown in FIG. 15B. The distance D between the light generation panel 1510 and the microlens array 1500 may be 300 μm (micrometers), although other distances may be used. An optically transparent material may fill the space between the light generation panel 1510 and the microlens array 1500 such that the light generation panel 1510 and the microlens array 1500 are physically connected to one another.

The microlens array 1500 and light generation panel 1510 of FIGS. 15A and 15B provide for microlens switched direction projection. Light emitted from the light generation panel 1510 can be steered after the microlens array 1500, directionally, by switching which location source it originates from on the light generation panel 1510. In this way, a particular source location can be chosen and the projection direction from the light exiting the microlens array 1500 can be steered.

In accordance with aspects of the invention, individual ones of the light emitting elements 1515 associated with a particular one of the lenses 1505 can be selectively turned on or off. As such, a first subset of the light emitting elements 1515 associated with a particular one of the lenses 1505 can be turned on concurrently with a second subset of the light emitting elements 1515 associated with a particular one of the lenses 1505 being turned off. Due to the different positions of the light emitting elements 1515 relative to the particular one of the lenses 1505 combined with the optical characteristics of the lens 1505 (e.g., index of refraction, focal length, etc.), a direction of light transmitted through the particular one of the lenses 1505 can be varied based on which one of the light emitting elements 1515 are included in the first subset (i.e., turned on) and which ones of the light emitting elements 1515 are included in the first subset (i.e., turned off) at any given time. In this manner, each one of the lenses 1505 and it's associated light emitting elements 1515 can be used to project light in a particular direction outward from the lens 1505.

This is demonstrated in the example shown in FIG. 15B in which: a first subset of light emitting elements 1515 behind lens 1505 is selected to emit light 1520 that, when refracted through lens 1505, exits lens 1505 in the direction of arrow 1525; a first subset of light emitting elements 1515 behind lens 1505′ is selected to emit light 1520′ that, when refracted through lens 1505′, exits lens 1505′ in the direction of arrow 1525′; a first subset of light emitting elements 1515 behind lens 1505″ is selected to emit light 1520″ that, when refracted through lens 1505″, exits lens 1505″ in the direction of arrow 1525″; and a first subset of light emitting elements 1515 behind lens 1505′″ is selected to emit light 1520′″ that, when refracted through lens 1505′″, exits lens 1505′″ in the direction of arrow 1525′″. The directions 1525, 1525′, 1525″, and 1525′ may each be different from one another, e.g., relative to the coordinate system defined by the x, y, and z axes shown in FIGS. 15A and 15B. In this manner, the light emitted from lenses 1505, 1505′, 1505″, and 1505′″ can be used project respective spots 1530, 1530′, 1530″, and 1530′″ at different locations. By properly selecting the first subset of light emitting elements 1515 for each of the lenses 1505 in a microlens array 1500, i.e., to achieve desired directions 1505, 1505′, 1505″, 1505′″, etc., the light generation panel 1510 and microlens array 1500 may be used to project an array 1535 of spots at a desired location. The greater the number of lenses 1505 in the microlens array 1500, the greater the resolution of the array 1535 of spots. The array 1535 of spots may thus create a pixelated image with each spot of the array 1535 of spots corresponding to a pixel of the image. In this manner, the light generation panel 1510 and microlens array 1500 may be used to project an image onto a desired location relative to the microlens array 1500. The image itself may be changed by appropriately selecting the first subset of light emitting elements 1515 for each of the lenses 1505 in a microlens array 1500. Additionally, or alternatively, the directions 1505, 1505′, 1505″, 1505′″, etc., may be changed, such that the location of the array 1535 of spots is changed, by appropriately selecting the first subset of light emitting elements 1515 for each of the lenses 1505 in a microlens array 1500. Each of the light emitting elements 1515 may be capable of emitting different colors of light such that the projected image is a color image. For example, each of the light emitting elements 1515 may comprise a red LED, a blue LED, and a green LED, each of which is individually controllable.

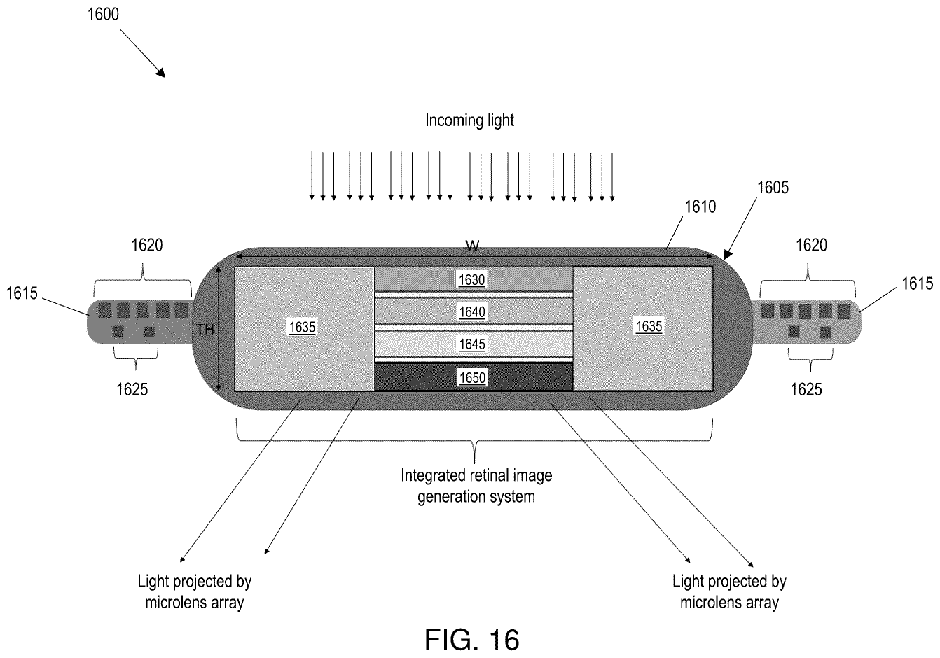

FIG. 16 shows an embodiment of a microlens device 1600 in accordance with aspects of the invention. The microlens device 1600 may be used as the devices 500/600/700 described herein. In embodiments, the microlens device 1600 includes a body 1605 that has a central portion 1610 and haptics 1615. The body 1605 may be made in the form of a single piece lens composed of materials such as acrylic and/or silicon lens material. In embodiments, the body 1605 comprises two haptics 1615 in the form of wings or tabs that each extend outward from the central portion 1610.

In embodiments, the microlens device 1600 comprises inductive coupling coils 1620, a wireless communication antenna 1625, an imaging system 1630, a power source 1635, control circuitry 1640, a light generation panel 1645, and a microlens array 1650. The light generation panel 1645 may correspond to light generation panel 1510 of FIGS. 15A and 15B, and the microlens array 1650 may correspond to microlens array 1500 of FIGS. 15A and 15B. In an exemplary embodiment, the inductive coupling coils 1620 and wireless communication antenna 1625 are embedded in one or both haptics 1615, and the remaining elements 1630, 1635, 1640, 1645, 1650 are integrated in chip stack contained in the body 1605. As shown in FIG. 16, the imaging system 1630 is at a first side of the chip stack such that it can receive incoming light from outside the eye, and the microlens array 1650 is at a second side of the chip stack opposite the first side of the chip stack such that the microlens array 1650 can project an image onto the retina inside the eye in which the microlens device 1600 device is implanted.

In embodiments, the imaging system 1630 receives incoming light from outside the eye and provides input to the control circuitry 1640 based on the received light, and the control circuitry 1640 provides electronic control signals to the light generation panel 1645 based on the input received from the imaging system 1630. Light emitted from the light generation panel 1645 can be steered via the microlens array 1650 directionally by switching which location source it originates on the light generation panel 1645. In this way, a particular source location can be chosen and the projection direction from the light exiting the microlens array 1650 can be steered. In this manner, a projection system comprising the light generation panel 1645 and the microlens array 1650 is controlled to reproduce an image received by the imaging system 1530 via projection onto the mapped areas of the retina. For example, the control circuitry 1640 may operate to receive one or more signals imaging system 1630 and to selectively energize respective subsets of light emitting elements in the light generation panel 1645, where each of the respective subsets of light emitting elements being associated with a respective one of the lenses in the microlens array 1650, to cause the light emitted by the respective subsets of light emitting elements to be refracted and transmitted in desired directions to project an array of spots at a desired location on the surface of the retina of the eye in which the microlens device 1600 is implanted. In this manner, the light generation panel 1645 and microlens array 1650 are configured to generate and project an image onto a retina of the eye in which the microlens device 1600 is implanted, the image being based on the light received by the imaging system 1630.

FIG. 16 shows an embodiment of the microlens device 1600 in which the imaging system 1630, power source 1635, control circuitry 1640, light generation panel 1645, and microlens array 1650 are arranged in four layers of a chip stack. However, embodiments are not limited to this configuration. Other arrangements of these elements in a chip or chip stack may be used so long as the imaging system 1630 is positioned to receive incoming light and the microlens array 1650 is positioned to project an image onto the retina inside the eye in which the microlens device is implanted.

The imaging system 1630 may comprise a CCD/imaging chip. The power source 1635 may comprise a battery that is rechargeable either via wired connection or wirelessly. The control circuitry 1640 may comprise a CMOS/analog/light generation panel control/wireless chip that is configured to control operation of the microlens device 1600. The light generation panel 1645 may comprise a light source/generation chip. The microlens array 1650 may comprise components of an array or plural individual microlenses.

The microlens device 1600 may be composed of sub-circuits which may be on disparate chip materials and made with disparate technologies, such as Si, InP, GaAs, Liquid Crystal, etc. This integrated system can be stacked in as shown in FIG. 16, with the connections between circuit elements being formed using BGA/C4/micro-BGA, through substrate (or silicon) vias (TSVs), and micro-TSVs. Physical connections between layers can be through solder or oxide bonding techniques.

In the microlens device 1600, sub-circuit chips may be thinned using wafer thinning techniques to be thin enough such that the entire system is such that the thickness dimension TH satisfies the expression 1 mm<=TH<=3 mm. These techniques are employed in stacked memory chips with wafers thinned to less than 20 μm thick and bonded to other wafers and connecting micro-TSVs are made between active layers that are 10 μm to 20 μm tall. The microlens device 1600 may be constructed such that the width dimension W satisfies the expression 1 mm<=W<=10 mm. A microlens device having these dimensions TH and W is suitable for implant in an eye, such as shown at FIGS. 5-7.

In the microlens device 1600, each sub circuit system made with a different material technology may be aligned and integrated such that they are on a same level as shown in the case of the optical source chips and retinal image generation chip. Additionally, the retinal image generation chip itself may consist of integrated subcomponents such as SOI chips, Liquid Crystal cavities, LEDs, etc.

In the microlens device 1600, the control circuitry 1640 may contain wireless communication circuitry such that the integrated system could be programmed externally. In embodiments, once the image mapping to healthy retinal tissue is programmed, the device does not need any wireless communication to produce a retinal image in the healthy regions of the retina. The wireless communication antenna(s) for this system could be in the chips themselves (e.g., in the control circuitry 1640) or can be co-fabricated in the haptics as shown at elements 1625.

In embodiments, the power source 1635 comprises a rechargeable battery that can be wirelessly recharged through inductive coupling using the inductive coupling coils 1620 and an external charging coil, such as those illustrated in FIGS. 14A and 14B.

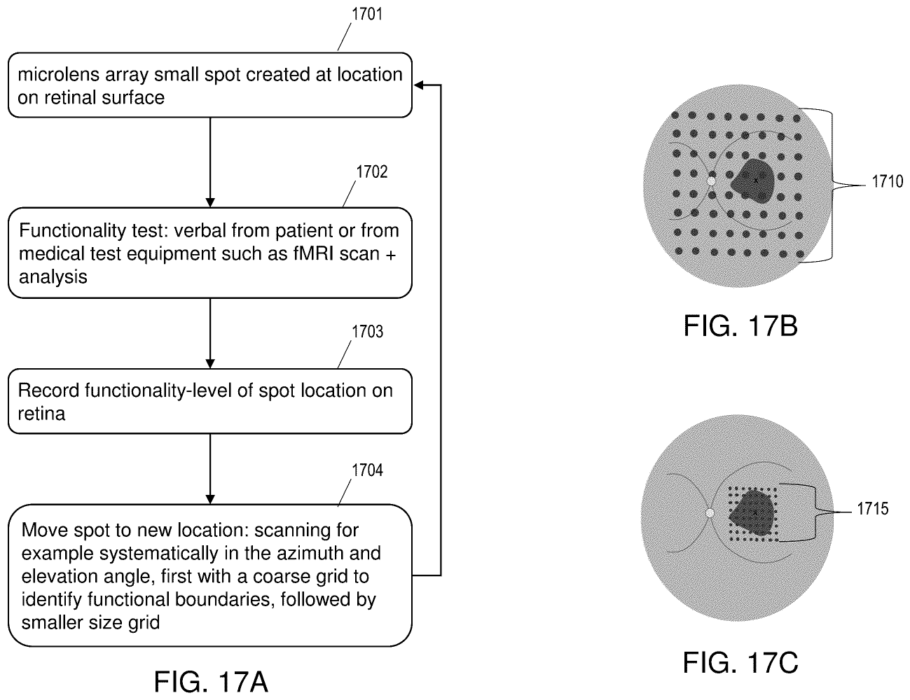

Still referring to FIG. 16, the imaging system 1630 may comprise any suitable type of on-chip imaging technology, such as a charge-coupled device (CCD). The imaging system 1630 may also include specialized local lens structures to enhance functionality of imaging chip. In embodiments, the output of the imaging system 1630 is a time-dependent electronic signal to control circuitry 1640. In embodiments, the control circuitry 1640 takes input of a time-dependent video signal, and an essentially fixed, but wirelessly programmable, mapping stored in memory (for example, in on-chip RAM) that is used to control where each pixel will be mapped to the retina surface. In embodiments, this programmable mapping is determined after diagnostic mapping as described, for example, at FIG. 17. During a programming phase, a wireless signal for the mapping is received through the wireless communication antenna 1625 which may be in the haptics and spiral around the center chipset as shown depicted in cross-section in FIG. 16.

FIG. 17 shows a flowchart of an exemplary method in accordance with aspects of the invention. The method may be carried out using the any of the microlens devices 500/600/700. At step 1701, the implanted microlens device projects an image on a small spot on the retina. At step 1702, the person indicates (e.g., via verbal feedback) whether they can or cannot see the spot. At step 1703, the system records the indication (yes or no) and the settings of the microlens device (e.g., settings of the light generation panel to selectively control selected ones of light emitting elements of the light generation panel). At step 1704, the microlens device changes the settings to change the direction of the projected image to a different location on the retina. The method then returns to step 1701 to repeat the projecting (step 1701), receiving feedback (step 1702), and recording (step 1703). By following this method and marching through plural discrete locations of projected pixels, the system can be used to create the mapping of which directions result in pixels being projected on a healthy part of the retina and which directions result in pixels being projected on a damaged part of the retina. In embodiments, this scanning is performed initially using a coarse grid 1710 (e.g., as shown in FIG. 17B) and then using a smaller sized grid 1715 (e.g., as shown in FIG. 17C). In embodiments, the mapping determined using this method is programmed to the control circuitry 1640 of the microlens device, e.g., using the wireless communication antenna 1625 as described herein. In embodiments, after being programmed, the mapping is used by the control circuitry 1640 of the microlens device to control the light generation panel 1645 to selectively energize ones of the light emitting elements relative to the microlens array 1650 such that light from the energized ones of the light emitting elements passes through the microlens array 1650 and is directed onto a healthy area of the retina.

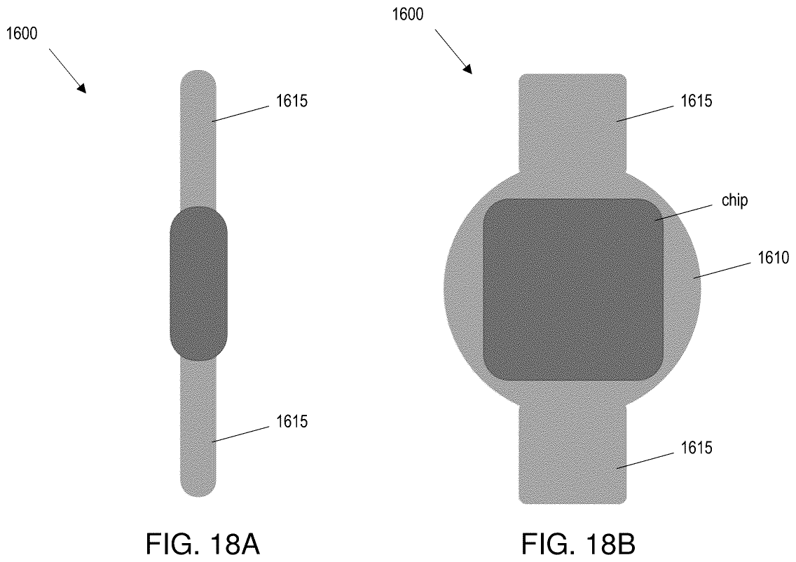

FIGS. 18A and 18B show a side view and a top view, respectively, of the microlens device 1600. As shown in FIGS. 18A and 18B, the microlens device 1600 includes a body comprising a center portion 1610 and haptics 1615. A chip (e.g., a stacked chip structure) containing the on-chip elements is disposed in the center portion 1610. The body may be composed of acrylic and/or silicone lens material, e.g., to form a single piece implantable lens replacement. The haptics 1615 allow for locating the microlens device 1600 in the center of the capsular bar or ciliary sulcus.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A device configured to be implanted in an eye, comprising:
an imaging system that receives visible light incoming to the eye; and
a light generation panel and a microlens array that are configured to generate and project an image onto a retina of the eye in which the device is implanted, the image being based on the light received by the imaging system.

2. The device of claim 1, further comprising control circuitry that causes the light generation panel and the microlens array to project the image onto a determined area of the retina.

3. The device of claim 2, wherein:
the microlens array comprises an array of optical lenses; and
the light generation panel comprises a plurality of individually controllable light emitting elements.

4. The device of claim 2, wherein the determined area of the retina is a healthy area of the retina.

5. The device of claim 4, wherein the control circuitry determines the determined area of the retina using a stored mapping.

6. The device of claim 5, wherein the imaging system, the control circuitry, the light generation panel, and the microlens array are arranged in a chip stack.

7. The device of claim 6, wherein:
the imaging system is at a first side of the chip stack; and
the microlens array is at a second side of the chip stack opposite the first side of the chip stack.

8. The device of claim 7, wherein:
the device comprises a body comprising a central portion and tabs extending outward from the central portion; and
the chip stack is in the central portion.

9. The device of claim 5, further comprising a wireless communication antenna that is configured to receive wireless communication signals from outside the device.

10. The device of claim 9, wherein the control circuitry is configured to program the mapping based on the wireless communication signals.

11. The device of claim 2, further comprising a rechargeable battery that is configured to power the imaging system, the control circuitry, and the light generation panel.

12. The device of claim 11, wherein the rechargeable battery is configured to be recharged wirelessly from a charging system located outside the eye.

13. The device of claim 1, wherein the device is configured to be implanted in a capsular bag of the eye.

14. The device of claim 1, wherein the device is configured to be implanted in a ciliary sulcus of the eye.

15. The device of claim 1, wherein the device is configured to be implanted in a chamber of the eye anterior to the iris.

16. A method comprising implanting the device of claim 1 into the eye.

17. A method of using the device of claim 1, the method comprising:
causing the device to project a diagnostic image on different locations of the retina of the eye;
receiving patient feedback for each of the different locations;
creating a mapping of the retina of the eye based on the feedback; and
programming the mapping into the device.

18. The method of claim 17, further comprising optimizing the mapping using artificial intelligence.

19. The method of claim 17, wherein the mapping maps the retina into functional areas and non-functional areas.

20. The method of claim 17, wherein the device is configured to control one or more elements of the light generation panel based on the mapping to project an image onto a functional area of the retina to reduce or eliminate a scotoma caused by a non-functional area of the retina.
